(19) United States
(12) Patent Application Publication
(10) Pub. No.: US 2007/0092871 A1
(43) Pub. Date: Apr. 26, 2007

(54) Microarray for pathogen identification

(75) Inventors: Michael J. Lodes, Seattle, WA (US); Dominic Suciu, Edmonds, WA (US)

Correspondence Address: COMBIMATRIX CORPORATION, 6500 HARBOUR HEIGHTS PARKWAY, MUKILTEO, WA, 98275, US

(73) Assignee: CombiMatrix Corporation

(21) Appl. No.: 11/584,379

(22) Filed: Oct. 20, 2006

Related U.S. Application Data
(60) Provisional application No. 60/728,641, filed on Oct. 20, 2005.

(51) Int. Cl. C12Q 1/70; C12Q 1/68; C12M 3/00

(52) U.S. Cl. 435/5; 435/6; 435/287.2; 977/924

(57) ABSTRACT

There is disclosed a microarray device for pathogen identification and for subtyping influenza A. Pools of primers are disclosed and used to amplify any subtype of influenza A. Pathogen identification includes influenza A, influenza B, parainfluenza virus, adenovirus, enterovirus, rhinovirus, human metapneumovirus, respiratory syncytal virus, herpes simplex viruses, SARS coronavirus, Epstein-Barr virus, human herpes virus, pan bacteria, Chlamydia, Mycoplasma, streptococcus, Bacillus anthracis, Streptococcuspyogenes, Mycoplasmapneumoniae, Chlamydiapneumoniae, Bacillus thuringiensis, Bacillus subtilis, Bacillus cereus, and B. anthracis. The probes are preferably selected from the first 500 nt of a gene from the 5′ end. The primers are preferably selected from bases in the 500 to 600 nt range from the 5′ end.

### STATEMENT REGARDING FEDERALLY SPONSORED RESEARCH

A portion of this invention was sponsored by Department of Defense Contract No. W911SR-04-C-0022.

### STATEMENT FOR A SEQUENCE LISTING

This application for patent incorporates by reference the accompanying compact disc (CD) having the title “1100 Sequence Listing.” The aforementioned CD has one file that has the title “1100.ST25.txt,” and has a size of 983 KB.

### TECHNICAL FIELD

Disclosed herein are microarrays for identifying pathogens. Specifically, provided herein are primers for pathogen amplification and oligonucleotide-containing microarrays having a plurality of probes for hybridization of pathogen targets. Using the primers and probes, an array is provided to determine whether an identified pathogen is present in a sample.

### BACKGROUND

Rapid and accurate detection and identification of pathogens has important applications in numerous fields including medicine, veterinary medicine, biology, and agriculture as well as within the military. For example, medical diagnosis of bacterial or viral infection requires identification of specific pathogens within clinical specimens followed by making a rational connection between the identified pathogen and the clinical syndrome. Similarly, diagnosis of a military biological agent or a clinical syndrome of military personnel requires identification of specific pathogens.

Current methods of pathogen diagnosis are often too long in duration (one or more days) and can be based on clinical symptoms, simple microscopic examination of tissue, immunological techniques such as immuno-fluorescense, immunoperoxidasae, ELISA, conventional culture, semiquantitative antigenemia assay, serological diagnosis, and PCR. Compared to other diagnosis methods, PCR provides increased detection sensitivity and can provide detection of several viruses in parallel. Parallel detection for discrete viruses can be accomplished by multiplexing specific primers, and parallel detection for members of a class can be accomplished through design of degenerate primers. However, PCR methods are not able to detect a wide variety of pathogens and pathogen subtypes in a single assay because designing multiplex primers for each pathogen or pathogen subtype is difficult and may be nearly impossible for rapidly mutating pathogens. Additionally, subtype identification may require additional lengthy and labor-intensive procedures such as sequencing, restriction enzyme analysis, and hybridization blotting. In general, current methods of diagnosis are relatively time consuming, labor-intensive and lengthy and are not capable of providing a high degree of accuracy in correct identification of specific pathogen or pathogen subtypes.

The common virus influenza A illustrates the problem of rapid mutation viral pathogen leading to difficulty in subtype identification, which can be of critical importance in diagnosis of a potentially epidemic or pandemic viral subtype, such as what has recently been termed “bird flu.” Influenza A virus is a negative strand RNA virus with a segmented genome that can infect a broad range of animals including man, horses, pigs, ferrets and various avian species. Identification of a virus subtype is typically by serological or molecular identification of the subtype of viral hemagglutinin (HA) and neuraminidase (NA) genes. Viruses with any combination of the 16 HA (Fouchier et al., J. Virol., 79:2814-2822, 2005) and 9 NA subtypes can infect aquatic birds while few subtypes have been found to infect humans. However, interspecies transmission can occur after recombination or mixing of subtypes in birds or pigs (Lipatov et al., J. Virol., 78:8951-8959, 2004; Scholtissek et al., Virology, 147:287-294, 1985; and Hoffmann et al., Arch. Virol., 146:1-15, 2001). In addition, new human strains of virus can arise by reassortment or antigenic shifts when two or more subtypes are circulating in the human population (Mizuta et al., Microbiol. Immunol., 47:359-361, 2003; Webby and Webster, Phil. Trans. R. Soc. London, B356:1815-1826, 2001). Re-emergence of a subtype into the human population can also occur by antigenic drift (Webby and Webster, Phil. Trans. R. Soc. London, B356:1815-1826, 2001), which occurs when genetic mutations of the HA and NA genes, from polymerase infidelity, creates virons that escape immune surveillance. For example, variability of the H3N2 subtype has required 19 changes in a vaccine component over 29 years (Hay et al., Phil. Trans. R. Soc. Lond. B356:1861-1870, 2001). New antigenic variants that require revisions in vaccine components can arise with a frequency of one per 1 to 2 years. Therefore, there is a need for diagnostic assays that are sensitive, specific as to serotype, and accurate. Additional benefits of less a labor-intensive and rapid test would be bonuses.

Identification of influenza subtypes is accomplished with viral detection (cell culture) and serological techniques such as complement fixation, hemagglutination, hemagglutination inhibition assays, and immunofluorescence methods (Allwinn et al., Med. Microbiol. Immunol., 191:157-160, 2002; Amano and Cheng, Anal. Bioanal Chem., 381:156-184, 2005; Palmer et al., Immunology Series No. 6. U.S. Dept. of Health, Education, and Welfare, p. 51-52, 1975; and Ueda et al., J. Clin. Microbiol. 36:340-344, 1998). However, even though traditional methods are generally effective, they involve the use of labor-intensive techniques and highly trained personnel. Because of their speed, specificity, and sensitivity, genomic assays are becoming more common for identifying the genotype of an unknown specimen (Taubenberger and Layne, Mol. Diagn., 6:291-305, 2001; Ellis and Zambon, Rev. Med. Virol., 12:375-389, 2002; and Zou, J. Clin. Microbiol. 35:2623-2627, 1997). Molecular methods also complement antigenic characterization in certain cases where antigenic tests are not specific enough to detect closely related groups (Schweiger et al., Med. Microbiol. Immunol., 191:133-138, 2002). Reverse transcription-polymerase chain reaction (RT-PCR) is used for virus identification (Hoffmann et al., Arch. Virol., 146:1-15, 2001; Adeyefa et al., Virus Res., 32:391-399, 1994; and Templeton et al., J. Clin. Microbiol., 42:1564-1569, 2004). However, a positive amplification can be verified only by subsequent assays to elaborate sequence information. By overcoming this limitation, microarrays and biosensors have become tools for viral discovery, detection and genotyping (Kessler et al., J. Clin. Microbiol., 42:2173-2185, 2004; Ivshina et al., J. Clin. Microbiol., 42:5793-5801, 2004; Sengupta et al., J. Clin. Microbiol., 41:4542-4550, 2003; Li et al., J. Clin. Microbiol., 39:696-704, 2001; Amano and Cheng, Anal. Bioanal. Chem., 381:156-184, 2005; Ellis and Zambon, Rev. Med. Virol., 12:375-389, 2002; and Wang et al., Proc. Biology, 1:257-260, 2003; Wang et al., Proc. Natl. Acad. Sci. USA, 99:15687-15692, 2002).

Unique oligonucleotide probe sequences are required to utilize the potential benefits of microarray-based assay of pathogens and subtypes of pathogens. Additionally, unique primer pools are required in order to amplify a target sample for a microarray-based assay. Unique subtypes of influenza A are of high interest because of the potential for a pandemic. Thus, there is a need in the art for oligonucleotide probes and primer pools capable of being used to amplify, detect, and distinguish a broad spectrum of pathogens and pathogen subtypes. The devices and methods disclosed herein address this need.

In the face of concerns over an influenza pandemic, identification of virulent influenza isolates must be obtained quickly for effective responses. Knowledge of the exact strain, origin of the strain, and probable characteristics of the virus are critical for surveillance of a disease outbreak and preventing the spread of the disease. Rapid subtype identification of flu is not always straightforward. Simple serological tests on infected individuals are awkward to administer and are an ineffective tool for monitoring viruses undergoing a high rate of mutation or rapid recombination. RT-PCR assays have better sensitivity but are problematic in scenarios where new strains of virus emerge or mixtures of viruses exist. RNA viruses such as flu undergo antigenic shift and genetic drift as they circulate through populations. Tracking these changes and keeping abreast of evolving viral variants is the key to effective vaccination and can provide insight as to why certain strains of flu are drug resistant or more lethal to infected hosts. In addition, influenza isolates circulating in non-human populations (e.g. birds, pigs, and dogs) must also be monitored on an ongoing worldwide basis to detect virulent isolates that have the potential to infect humans directly or recombine with common human strains of flu to produce lethal hybrids. In many situations, the identification of the circulating subtype (e.g. by simple serotype or a simple RT-PCR test) is not sufficient, and specific knowledge of the genetic makeup of the virus is required. For example, the avian H5N1 virus has significant potential for further recombination with common human strains (e.g. H3N2) or other non-human strains common in avian populations (H7 and H9 strains). The H5N1 subtype is also difficult to identify because of the lack of sensitivity and specificity of many of the commercial tests. In addition, genotype Z, the dominant H5N1 virus genotype circulating in Vietnam and Thailand contains a mutation that is associated with resistance to amantadine and rimantadine. Because of the high susceptibility in humans and resistance to antibiotics of this isolate, neuraminidase inhibitors must be given within 48 hours of onset of illness to be effective. Thus rapid and specific identification of this subtype and accurate sequence information is crucial for proper treatment.

### SUMMARY

Disclosed herein are microarrays for genetic identification of upper respiratory pathogens comprising: a microarray device having a plurality of oligonucleotide probe sequences, wherein the oligonucleotide probe sequences correspond to at least five unique genes and distinct sequence regions of pathogen genomes selected from the group consisting of influenza A (SEQ ID NO:1-11, 251-261, 495-505, 726-736), influenza B (SEQ ID NO:12-35, 262-285, 506-525, 737-758), parainfluenza virus (SEQ ID NO:36-70, 286-315, 526-560, 759-792), adenovirus (SEQ ID NO:71-95, 316-341, 561-582, 793-818), enterovirus (SEQ ID NO:96-121, 342-368, 583-604, 819-842), rhinovirus (SEQ ID NO:122-138, 369-385, 605-621, 843-859), human metapneumovirus (SEQ ID NO:139-141, 386-388, 622-624, 860-862), respiratory syncytal virus (SEQ ID NO:142-177, 389-425, 625-657, 863-896), herpes simplex viruses (SEQ ID NO:178-184, 426-431, 658-665, 897-905), SARS coronavirus (SEQ ID NO:185-202, 432-448, 666-682, 906-924), Epstein-Barr virus (SEQ ID NO:203-204, 449, 925-927), human herpes virus (SEQ ID NO:205-206,450-451, 863, 928-929), pan bacteria (SEQ ID NO:207-210, 452-454, 684-687, 930-933), Chlamydia (SEQ ID NO:211-226, 455-470, 688-701, 934-949), Mycoplasma (SEQ ID NO:227-238,471-482, 702-713, 950-962), streptococcus (SEQ ID NO:239-244, 483-488, 714-719, 963-968), Bacillus anthracis (SEQ ID NO:245-250, 489-494, 720-725, 969-97), Streptococcuspyogenes (SEQ ID NO:975-1013), Mycoplasmapneumoniae (SEQ ID NO:1014-1051), Chlamydiapneumoniae (SEQ ID NO:1052-1083), Bacillus thuringiensis (SEQ ID NO:1084-1115), Bacillus subtilis (SEQ ID NO:1116-1154), Bacillus cereus (SEQ ID NO:1155-1157), and B. anthracis (SEQ ID NO:1158-1173), and combinations thereof.

Preferably, the oligonucleotide probe sequences are selected in a range from 400 to 800 bases on each gene of each pathogen. More preferably, the oligonucleotide probe sequences are selected in a range from 400 to 800 bases on a gene of each pathogen at a 5′ end.

Also disclosed herein is a microarray for subtyping influenza A comprising: a solid surface having a plurality of known oligonucleotide probe sequences, wherein the oligonucleotide probes correspond to at least five subtypes of influenza A selected from the group consisting of H1 (SEQ ID NO:1174-1573), H2 (SEQ ID NO:1574-1973), H3 (SEQ ID NO:1975-2373), H4 (SEQ ID NO:2374-2573), H5 (SEQ ID NO:2574-2973), H6 (SEQ ID NO:2974-3369), H7 (SEQ ID NO:3370-3769), H8 (SEQ ID NO:3770-3887), H9 (SEQ ID NO:3888-4287), H10 (SEQ ID NO:4288-4390), H11 (SEQ ID NO:4391-4486), H12 (SEQ ID NO:4487-4587), H13 (SEQ ID NO:4588-4705), H14 (SEQ ID NO:4706-4761), H15 (SEQ ID NO:4762-4807), N1 (SEQ ID NO:4808-5207), N2 (SEQ ID NO:5208-5607), N3 (SEQ ID NO:5608-5878), N4 (SEQ ID NO:5879-5920), N5 (SEQ ID NO:5921-5995), N6 (SEQ ID NO:5996-6116), N7 (SEQ ID NO:6117-6216), N8 (SEQ ID NO:6217-6561), and N9 (SEQ ID NO:6562-6661) and combinations thereof.

Preferably, the oligonucleotide probes are selected in a range from 400 to 800 bases on a gene of each pathogen. More preferably, the oligonucleotide probes are selected in a range from 400 to 800 bases on a gene of each pathogen at a 5′ end.

Further still, the present invention provides a pool of primers for amplifying influenza A comprising a primer set selected from the group consisting of SEQ ID NO:6662-6699 and combinations thereof. Preferably, the primer set is selected in a range from 50 to 200 bases of HA and NA genes. More preferably, the primer set is selected in a range from 450 to 700 bases from a 5′ end of HA and NA genes.

Further still, the present invention provides a pool of primers for amplifying influenza A comprising a primer set selected from the group consisting of SEQ ID NO:6700-6731 and combinations thereof. Preferably, the primer set is selected in a range from a 50 to 200 bases of HA and NA genes. More preferably, the primer set is selected in a range from 450 to 700 bases from a 5′ end of HA and NA genes.

### BRIEF DESCRIPTION OF THE DRAWINGS

FIG. 1 shows an image of gels taken on PCR samples after amplification of known influenza A subtypes using reverse primer pools. Four pools of reverse primers were used: HA-universal (U), NA-universal (U), HA-degenerate (D), and NA-degenerate (D). Arrowheads indicate the approximate range of the sizes predicted for the amplicons.

FIG. 2 shows an image of gels taken on PCR samples after amplification of unknown influenza A subtypes using reverse primer pools. Four pools of reverse primers were used: HA-universal (U), NA-universal (U), HA-degenerate (D), and NA-degenerate (D). Arrowheads indicate the approximate range of the sizes predicted for the amplicons.

FIG. 3 shows an image of gels taken on PCR samples after amplification using literature primers. Amplification was performed on full-length hemagglutinin (HA) and neuraminidase (NA) (1700 bp and 1400 bp respectively) with published universal influenza A (InA) primers.

FIG. 4 shows the results of hybridization of an H1N1 influenza A sample to a microarray having probes for pathogens.

FIG. 5 shows the results of hybridization of an influenza B sample to a microarray having probes for pathogens.

FIG. 6 shows plots of influenza A subtype identification of the HA gene.

FIG. 7 show plots of influenza A subtype identification of the NA gene.

### DETAILED DESCRIPTION

Microarray preparation methods for making oligonucleotide probes for pathogen identification include the following: (1) spotting a solution on a prepared surface using spotting robots; (2) in situ synthesis by printing reagents via ink jet or other computer printing technology and using phosphoramidite chemistry; (3) in situ parallel synthesis using electrochemically generated acid for removal of protecting groups and using standard phosphoramidite chemistry; (4) in situ synthesis using maskless photo-generated acid for removal of protecting groups and using regular phosphoramidite chemistry; (5) mask-directed in situ parallel synthesis using photo-cleavage of photolabile protecting groups (PLPG) and phosphoramidite chemistry; (6) maskless in situ parallel synthesis using PLPG and digital photolithography and standard phosphoramidite chemistry; and (7) electric field attraction/repulsion for depositing fully formed oligonucleotides onto known locations.

An electrode microarray for in situ oligo synthesis using electrochemical deblocking is disclosed in Montgomery U.S. Pat. Nos. 6,093,302; 6,280,595, and 6,444,111 (Montgomery I, II, and III respectively), all of which are incorporated by reference herein. Another and materially different electrode array (not a microarray) for in situ oligo synthesis on surfaces separate and apart from electrodes using electrochemical deblocking is disclosed in Southern U.S. Pat. No. 5,667,667, which is incorporated by reference herein. Photolithographic techniques for in situ oligo synthesis are disclosed in Fodor et al. U.S. Pat. No. 5,445,934 and the additional patents claiming priority thereto, all of which are incorporated by reference herein. Electric field attraction/repulsion microarrays are disclosed in Hollis et al. U.S. Pat. No. 5,653,939 and Heller et al. U.S. Pat. No. 5,929,208, both of which are incorporated by reference herein. A review of oligo microarray synthesis is provided by: Gao et al., Biopolymers 2004, 73:579.

One method making primers for amplifying pathogens in a sample for pathogen identification is standard phosphoramidite synthesis on beads in a column. Primers are a collection of oligonucleotides synthesized by standard means and often can be ordered through various commercial sources.

The oligonucleotide probe designs and primer sequence designs isolate the most distinct sequence regions of the genome of the selected list of pathogens and their various strains. This approach to design provides the optimal sequences to enable a microarray diagnostic device that can identify and distinguish pathogens in a sample and distinguish strains of the pathogens.

DNA microarray device is used to detect and accurately type flu strains (influenza) with hemeagglutinin subtypes 1 through 15 and neuraminidase subtypes 1 through 9 using a protocol that requires less than four hours start to finish. In addition to providing very high resolution genotype information on any given flu strain, the device can also provide information on novel strains of flu produced by rapid mutation or recombination between multiple strains of flu.

Reference, clinical, and military laboratories must evaluate antigenic shift and genetic drift in the common flu strains circulating each year so that these changes can be addressed in vaccine development. Also, avian influenza isolates must be monitored on a worldwide basis to detect virulent isolates that have the potential to infect humans and produce a future epidemic or pandemic. In many situations, the identification of the circulating subtype is not sufficient, and a specific gene sequence is required. For example, there is an increased risk for spread in the human population of the highly virulent avian H5N1 virus because of the combination of the large number of poultry farms, the potential for recombination with a human strain, and the high susceptibility to infection with this strain in the human population (Yuen and Wong, Hong Kong Med. J., 11:189-99, 2005; Chen et al., Proc Natl. Acad. Sci. USA 101:10452-10457, 2004). The H5N1 subtype is difficult to identify because of the lack of sensitivity and specificity of many of the commercial tests. In addition, genotype Z, the dominant H5N1 virus genotype that is circulating in Vietnam and Thailand, contains a mutation that is associated with resistance to amantadine and rimantadine (Yuen and Wong, Hong Kong Med. J., 11:189-99, 2005). Because of the high susceptibility in humans and resistance to antibiotics of this isolate, neurarninidase inhibitors must be given within 48 hours of onset of illness to be effective. Thus rapid and specific identification of this subtype and accurate sequence information is crucial for proper treatment.

While traditional assays for influenza detection and typing represent the current standard, these assays cannot meet the future needs of rapid, sensitive, specific, and simple methods. Herein is provided a specific influenza A microarray diagnostic device that contains specific probes for each of the 15 HA subtypes and 9 NA subtypes. Hybridizable target has been generated for all 15 HA and 9 NA subtypes by PCR amplification of influenza A reference strains. Non-overlapping probes with similar annealing stabilities were generated from the influenza sequence database, consisting of over 3000 HA and over 1000 NA sequences. Subtype-specific probes were then selected from a pool of over 23,000 HA and 15,000 NA sequences and then compared to the database to insure that each probe was unique.

HA and NA subtypes were correctly identified with this assay platform. Weak or average intensity profiles for some subtypes were due to dilution of positive signal by subtype probes that were not hybridized or weakly hybridized to the matching subtype target. By eliminating unnecessary or cross-reacting probes and limiting probe number to approximately 100 for each subtype, this artifact of the array can be corrected. A second approach is to subdivide the probes for each subtype into similar groupings and thus concentrate the positive probes; this approach increases the average signal for a positive identification. This approach also produces positive probe sequences that are tiled across the viral sequence of interest and results in an approximation or a best-fit sequence for the unknown subtype.

The inventive microarray diagnostic device presented here, was able to sequence approximately 500 or more nucleotides from the HA and NA genes from isolates that have been identified with the illustrative microarray using the same target preparation for both assays. The inventive microarrays contain either a consensus subtype sequence or a known subtype sequence that lacks a high degree of secondary structure.

Sequencing probes were tiled by one nucleotide across the sequence of interest, and for each nucleotide that was interrogated, four probes were designed that differed only at the 5′ end. After the enzymatic extension of a common labeled primer, only the perfect match probe sequence was ligated to the labeled primer. The resulting sequence was automatically extracted for database searches.

Influenza A HA subtypes 1 through 15 and NA subtypes 1 through 9 were rapidly and specifically identified and sequenced using the illustrated oligonucleotide microarrays with a protocol that required less than one hour for target hybridization. The inventive assay precluded the need for traditional target labeling systems and integrated an enzyme-based procedure that overcame many of the shortfalls of traditional thermal hybridizations such as optimal hybridization conditions and difficult mismatch detection (Sengupta et al., J. Clin. Microbiol. 41:4542-4550, 2003). However, the illustrated diagnostic microarray device can also be used with traditional microarray labeling, hybridization, and washing protocols.

The oligonucleotide sequencing probe sequence “content” on the diagnostic microarray device was based upon several criteria, including but not limited to, TM (melting temperature), length, and location in the pathogen target gene sequences; for example, the HA and NA gene sequences for different influenza strains. Published structural analysis and antigenic epitope mapping indicated that the HA receptor-binding structure and surface antigenic epitopes are located within the 5′ 720 bp (approx. bp 370 to 720) for subtype H9 and the 5′ 600 bp (approx. bp 180 to 610) for subtypes H3 and H5. The mapping of these sites was based on the selection of escape mutants with a panel of monoclonal antibodies (Kaverin et al., J. Gen Virol. 83:2497-2505, 2002; Kaverin et al., J. Virol. 78:240-249, 2004; and Gulati et al, J. Virol. 76:12274-12280, 2002).

The microarray device herein provided an ability for rapid identification of the HA and NA subtypes followed by sequence from critical regions of the HA and NA genes, such as surface antigenic epitopes, and significantly decreased the time and cost for the identification of potential lethal virus (upper respiratory virus) strains. The detection, identification, and sequencing of viral genomes in samples using oligonucleotide microarray technology is a viable rapid approach and can complement or supplant traditional methods.

### EXAMPLE 1

In this example, samples of influenza A were amplified using primers of the present invention and disclosed primers. The reverse primers of the present invention include pool #1 for amplifying subtype H1-H15 (SEQ ID NO:6662-6684), pool #2 for amplifying subtype N1-N9 (SEQ ID NO:6685-6699), pool #3 for amplifying subtype H1-H15 (SEQ ID NO:6700-6720), and pool #4 for amplifying subtype N1-N9 (SEQ ID NO:6721-6731). The primers were chosen from the 5′ 500 to 600 nucleotide region of the HA and NA genes. The forward primer for first stage cDNA was a universal primer having the following sequence: taatacgact cactatagga gcaaaagcag g (SEQ ID NO:6732). The tag of the sequence is underlined and is a T7 oligo, and the universal influenza sequence is shown in bold letters. The universal influenza sequence is complimentary to the 12 bp of the 5′ end of the HA and NA genes. The forward primer for second stage amplification was a T7 universal primer having the following sequence: gcatcctaat acgactcact atagg (SEQ ID NO:6733). The samples amplified by these primers were two H1N1 subtypes, an H2N2 subtype, and three unknown subtypes.

The primers used from the literature were for full-length amplification of the HA and NA genes. The universal forward primer sequence was aggagcaaaagcagg (SEQ ID NO:6734). The universal reverse primer sequence was ggagtagaaacaagg (SEQ ID NO:6735). The universal HA specific forward primer sequence was aggagcaaaagcagggg (SEQ ID NO:6736). The universal HA specific reverse primer sequence was agtagaaacaagggtgtttt (SEQ ID NO:6737). The universal NA specific forward primer sequence was aggagcaaaagcaggact (SEQ ID NO:6738). The universal NA specific reverse primer sequence was agtagaaacaaggagtttttt (SEQ ID NO:6739). Underlined bases are linker bases. Non-underlined bases are influenza A. The sample amplified by these primers was a H9N2 subtype.

For all samples amplified using primers, the following procedure was used. First-strand cDNA was produced from influenza virus RNA sample using SuperScript® II reverse transcriptase (Invitrogen Corporation) and the forward primer for the first stage cDNA. A solution was made comprising 3 μl of distilled water, 7 μl of viral RNA solution, 1 μl or primer, and 1 μl of dNTP solution. This solution was held at 70° C. for 5 minutes and then placed on ice. To this solution was added 4 μl of 5× first strand buffer, 2 μl of 0.1 molar DTT, 0.5 μl of RNAse inhibitor, and 0.5 μl of distilled water. The program for amplification was as follows: 40° C. for 2 minutes, addition of 0.5 μl of SuperScript® II, 40° C. for 48 minutes, 50° C. for 50 minutes, 70° C. for 15 minutes, and 4° C. until the next step.

For PCR amplification, the following solution was made for each primer set of the present invention: 67 μl of distilled water, 10 μl of 10× polymerase buffer, 10 μl of DMSO, 1 μl of Taq DNA polymerase, 3 μl of a 10 millimolar dNTP mix, 2 μl of a 10 micromolar T7 forward primer, 2 μl of a 10 micromolar specific reverse primer or in the case of unknown samples 2 μl of a pool of 100 μM of primers, and 5 μl of first-strand cDNA. The reaction conditions consisted of a 5 min denaturation at 94° C., followed by 40 cycles of a 30 sec, 94° C. denaturation step; a 30 sec, 55° C. annealing step; and a 30 sec, 72° C. extension; and finally a 10 min extension at 72° C. The resulting PCR product was cleaned with a Qiagen QIAquick nucleotide removal kit (#28306) and eluted in 100 μl of distilled water. A second, one-way amplification resulted in single stranded target. One-way amplifications were accomplished with 69 μl of distilled, deionized water, 10 μl of 10× polymerase buffer, 10 μl of DMSO, 1 μl of Taq DNA polymerase, 3 μl of a 10 μM dNTP mix, 2 μl of a 10 μM specific reverse primer and 5 μl of cleaned amplification product from amplification 1. The reaction conditions consisted of a 5 min denaturation at 94° C., followed by 50 cycles of a 30 sec, 94° C. denaturation, a 30 sec, 55° C. annealing, and a 30 sec, 72° C. extension; and finally a 10 min extension at 72° C. The resulting product was purified with a Qiagen QIAquick Nucleotide Removal kit and eluted in 100 μl of distilled water. This step resulted in tagged, single-stranded target for hybridization.

For all samples amplified using the literature primers, the following procedure was used. The following were added to a 0.2 ml tube: 2 microliters of RNA sample and 2 microliters of forward primers (10 mM). The tube was gently mixed. The tube was then heated to 70° C. for 5 minutes and then placed on ice. A solution was made comprising 8.8 microliters of distilled water, 4.0 microliters of 5× SSII buffer, 1 microliter of dNTP mix (10 mM each), 2 microliters of 100 mM DTT, and 0.2 microliters of SuperScript II RT (200 U/microliter). This solution was added to the tube and mixed gently. The heat cycle comprised: 42° C. for 10 minutes, 50° C. for 50 minutes, 70° C. for 15 minutes, and then held at 4° C.

For PCR amplification, the following solution was made for each of the literature primers: 67 microliters of distilled water, 10 microliters of 10× PCR buffer, 10 microliters of DMSO, 3 microliters of dNTP (10 mM each), 2 microliters of forward primer (10 micromolar), 2 microliters of reverse primer (10 micromolar), 5 microliters of RT template, and 1 microliter of Taq polymerase (added after other components were mixed and heated to 95° C.) The heat cycling was as follows: 1 cycle at 95° C. for 5 minutes; 40 cycles of 95° C. for 30 sec, 55° C. for 60 sec, and 72° C. for 2 min; 1 cycle at 72° C. for 10 min; and 1 cycle at 4° C. until ready for next step. The PCR products were purified using a Qiagen QIAquick Nucleotide Removal kit (#28306). Elution was done using 100 microliters of distilled water. Single-stranded DNA was isolated as follows: Streptavidin Magnetic Beads (NEB S1420S) were washed three times using 2× PBS. To 100 microliters of the cleaned PCR product, 24 microliters of 10× PBS was added and then mixed with the beads. The mixture was incubated for 15 min with frequent mixing. The mix was spun at 6000 RPM for 1 min and then washed two times with 2× PBS. The supernatant was removed and 20 microliters of 0.1 M NaOH was added. This solution was incubated for 10 minutes at room temperature. The solution was spun, and the supernatant was recovered. Another 20 microliters of 0.1 M NaOH was added followed by another 10 minute incubation. The solution was spun and supernatant recovered. To the combined supernatants, 20 microliters of 0.2 M HCl was added. Then, 6 microliters of 10× PBS was added, and the solution was mixed. The resulting solution was purified using a Qiagen QIAquick Nucleotide Removal kit (#28306). Elution was done using 100 microliters of distilled water.

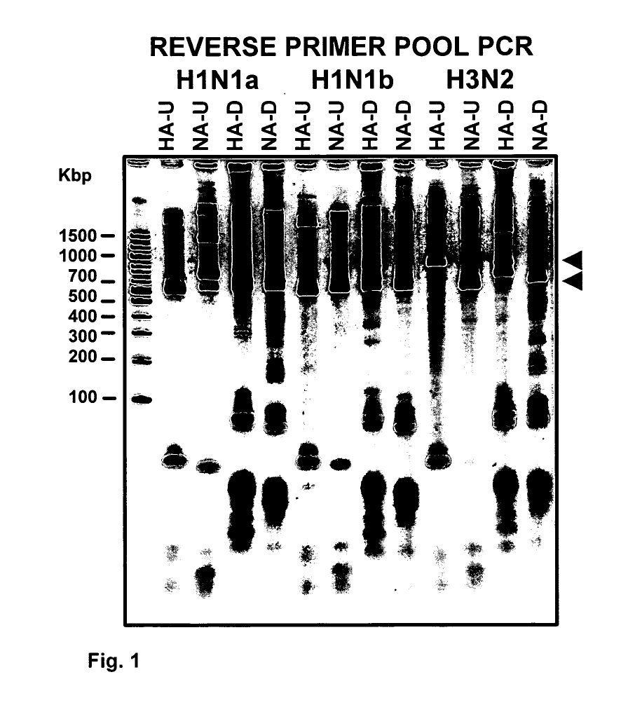

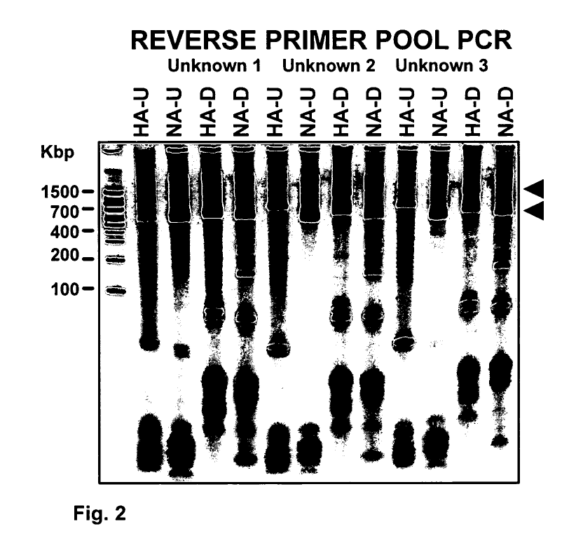

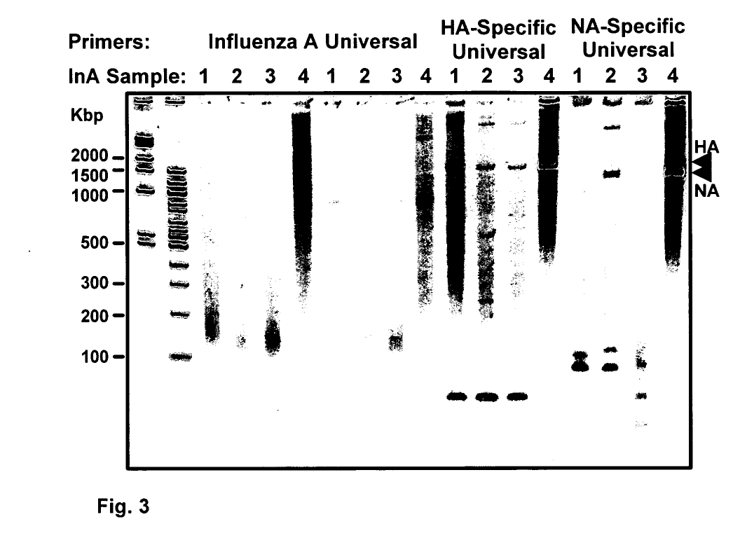

The resulting PCR products were run on gel electrophoresis. Images of the gel are shown in FIGS. 1-3. FIG. 1 demonstrates that the reverse primer pools amplified the two H1N1 subtypes and the H3N2 subtype as evidenced by strong bands at the expected size range. Pool #1 (HA-U), Pool #2 (NA-U), Pool #3 (HA-D), and Pool #4 (NA-D) showed strong bands for each subtype. FIG. 2 shows that the reverse primer pools of the present invention amplified the three unknown samples. Each primer pool of the present invention showed strong bands. Pool #1 appeared to be the weakest of the pools for unknown 1 but still showed evidence of amplification. FIG. 3 demonstrates that the (non-inventive) reverse primers used from the literature for full strand amplification did not amplify the four different H9N2 subtypes adequately. The universal forward and reverse primers showed almost no amplification. The HA-specific primers showed some amplification, especially for sample 4. The NA-specific primers showed minimal amplification only for samples 1 and 4.

### EXAMPLE 2

In this example, a microarray was prepared having probes of the present invention to identify the following pathogens: pan influenza A, (SEQ ID NO:1-11, 251-261, 495-505, 726-736), p (subtypes H1-H15 and subtypes N1-N9), pan influenza B (SEQ ID NO:12-35, 262-285, 506-525, 737-758), parainfluenza virus (SEQ ID NO:36-70, 286-315, 526-560, 759-792), adenovirus (SEQ ID NO:71-95, 316-341, 561-582, 793-818), enterovirus (SEQ ID NO:96-121, 342-368, 583-604, 819-842), rhinovirus (SEQ ID NO:122-138, 369-385, 605-621, 843-859), human metapneumovirus (SEQ ID NO:139-141, 386-388, 622-624, 860-862), respiratory syncytal virus (SEQ ID NO:142-177, 389-425, 625-657, 863-896), herpes simplex viruses (SEQ ID NO:178-184, 426-431, 658-665, 897-905), SARS coronavirus (SEQ ID NO:185-202, 432-448, 666-682, 906-924), Epstein-Barr virus (SEQ ID NO:203-204, 449, 925-927), human herpes virus (SEQ ID NO:205-206, 450-451, 863, 928-929), pan bacteria (SEQ ID NO:207-210, 452-454, 684-687, 930-933), Chlamydia (SEQ ID NO:211-226, 455-470, 688-701, 934-949), Mycoplasma (SEQ ID NO:227-238, 471-482, 702-713, 950-962), streptococcus (SEQ ID NO:239-244, 483-488, 714-719, 963-968), Bacillus anthracis (SEQ ID NO:245-250, 489-494, 720-725, 969-97), Streptococcuspyogenes (SEQ ID NO:975-1013), Mycoplasmapneumoniae (SEQ ID NO:1014-1051), Chlamydiapneumoniae (SEQ ID NO:1052-1083), Bacillus thuringiensis (SEQ ID NO:1084-1115), Bacillus subtilis (SEQ ID NO:1116-1154), Bacillus cereus (SEQ ID NO:1155-1157), and B. anthracis (SEQ ID NO:1158-1173.

Additionally, the microarray had subtyping sequences for influenza A as follows: H1 (SEQ ID NO:1174-1573), H2 (SEQ ID NO:1574-1973), H3 (SEQ ID NO:1975-2373), H4 (SEQ ID NO:2374-2573), H5 (SEQ ID NO:2574-2973), H6 (SEQ ID NO:2974-3369), H7 (SEQ ID NO:3370-3769), H8 (SEQ ID NO:3770-3887), H9 (SEQ ID NO:3888-4287), H10 (SEQ ID NO:4288-4390), H11 (SEQ ID NO:4391-4486), H12 (SEQ ID NO:4487-4587), H13 (SEQ ID NO:4588-4705), H14 (SEQ ID NO:4706-4761), H15 (SEQ ID NO:4762-4807), N1 (SEQ ID NO:4808-5207), N2 (SEQ ID NO:5208-5607), N3 (SEQ ID NO:5608-5878), N4 (SEQ ID NO:5879-5920), N5 (SEQ ID NO:5921-5995), N6 (SEQ ID NO:5996-6116), N7 (SEQ ID NO:6117-6216), N8 (SEQ ID NO:6217-6561), and N9 (SEQ ID NO:6562-6661).

The microarray was a CombiMatrix CustomArray™ 12k microarray, which was used to synthesize the DNA probes using electrochemical synthesis. The microarray had approximately 12,000 platinum electrodes on a surface having a porous reaction layer. Each electrode was electronically addressable via computer control. The probes were electrochemically synthesized in situ onto known locations associated with the electrodes on the microarray. The known locations were each a volume within the porous reaction layer located over the electrodes. The porous reaction layer was composed of a sucrose matrix. The electrochemical synthesis used phosphoramidite chemistry coupled with electrochemical deblocking of the protecting groups in a confined manner on the synthesized DNA for the addition of each subsequent nucleotide. For bonding of the phosphoramidites, the microarray had organic reactive hydroxyl groups provided by the sucrose. Electrochemical deblocking involved biasing an electrode with current as an anode to generate acidic conditions within a defined volume of the porous reaction layer matrix at the electrode that were sufficient to remove the protecting group only within the defined volume of the porous reaction layer matrix located at the active electrode. Buffer in the solution used for deblocking and natural diffusion prevented deblocking at non-activated electrodes; in other words, the buffer and the resistance associated with natural diffusion confined the region of acidic conditions to the confined volume of the porous reaction layer. Removal of the protecting group allowed addition of the next phosphoramidite.

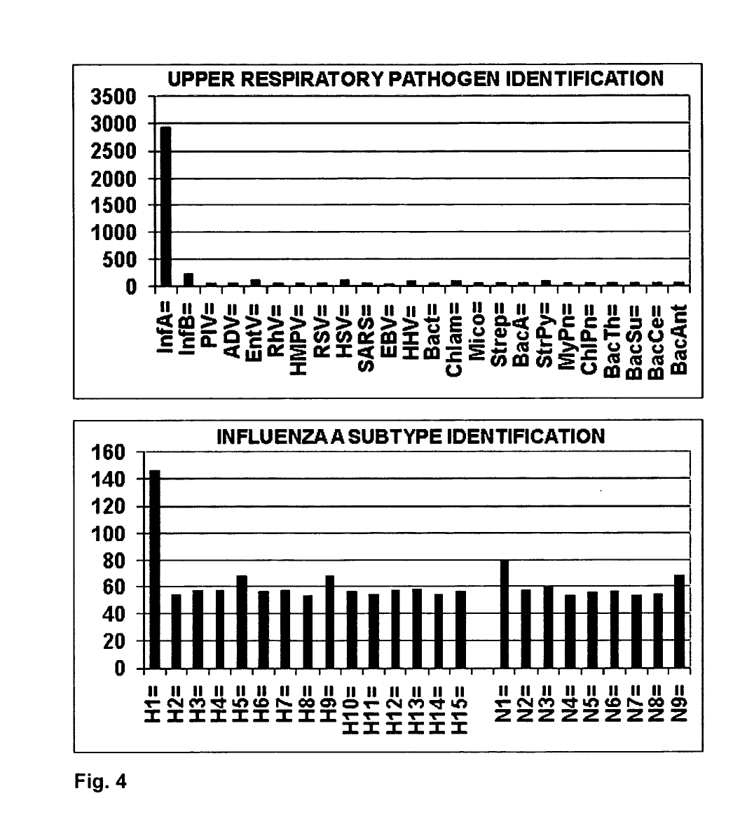

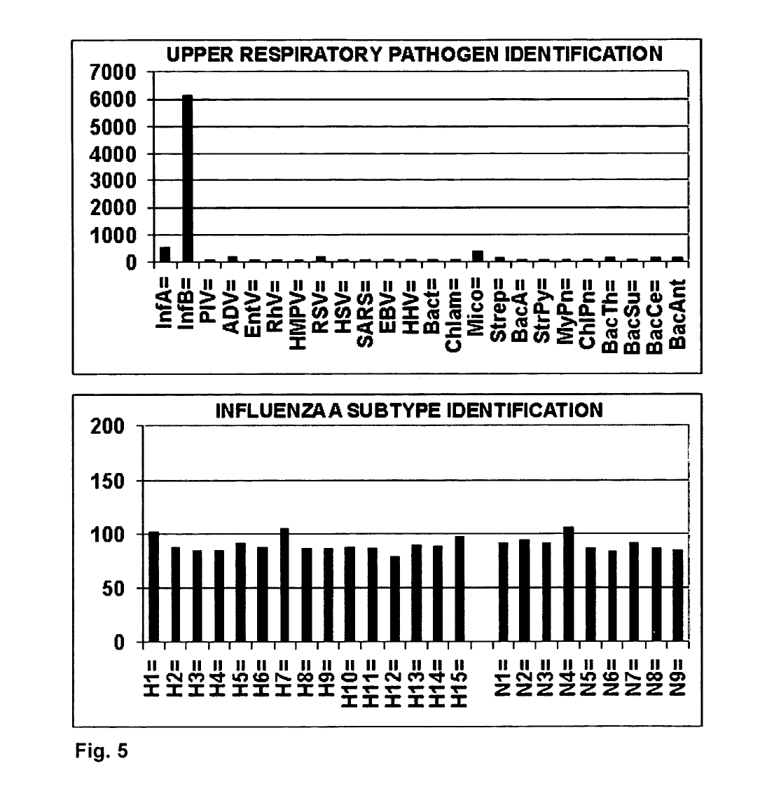

After synthesis of all probes, the microarray was exposed to a target sample to allow hybridization of pathogens to the probes. FIG. 4 shows fluorescence versus pathogen type and influenza A subtype for an H1N1 influenza A sample. As expected, the pan influenza A has higher fluorescence as well as the H1 and the N1 subtype probes. FIG. 5 shows fluorescence versus pathogen type and influenza A subtype for an influenza B sample. As expected, the pan influenza B probes show higher fluorescence, and the subtyping of influenza A does not show any increase in fluorescence of the subtype probes.

### EXAMPLE 3

In this example, microarrays were prepared as in Example 2 except that the microarrays only had probes for influenza A subtyping. Additionally, the microarrays had probes for hybridization and for hybridization followed by extension and ligation of hybridized primers to tagged targets. The extension ligation method is detailed in U.S. patent application Ser. No. 11/110,630 filed 20 Apr. 2005, the disclosure of which is incorporated by reference herein. Briefly, the method comprises (1) providing a microarray device having a plurality of oligonucleotide probes attached thereto, wherein each probe has a terminal nucleotide that is complementary to a target nucleotide; (2) forming a plurality of hybridized structures on the microarray, wherein each hybridized structure is formed by contacting the microarray under a hybridizing condition to a hybridizing solution comprising a plurality of tagged targets and a plurality of detection sequences, wherein each hybridized structure comprises one tagged target hybridized to one probe and to one detection sequence; (3) extending each hybridized structure using an extension-ligation solution; (4) removing non-bound material by washing the microarray; and (5) identifying the target nucleotide and a hybridized sequence from the hybridized structures having ligation. The tag on the targets is preferably added during amplification. The plurality of tagged targets is selected from the group consisting of tagged target DNA and tagged target RNA, and combinations thereof. The tagged target DNA may be a cDNA. The tagged target RNA may be an mRNA. The plurality of tagged targets may be first amplified. Preferably, the amplification is by PCR. Preferably, the hybridizing solution comprises a plurality of tagged targets and a plurality of detection sequences in a buffer solution comprising a 1×T4 ligase buffer. Preferably, the hybridizing condition comprises approximately 45° C. for approximately one hour. Preferably, the extension-ligation solution comprises water, buffer, triphosphate mix, polymerase, and ligase. Preferably, the extension-ligation condition comprises incubation of the microarray exposed to the extension-ligation solution at approximately thirty-seven degrees centigrade for approximately one hour. The polymerase is selected from the group consisting of DNA polymerase and RNA polymerase, and combinations thereof. Preferably, the polymerase is selected from the group consisting of Taq polymerase Stoffel fragment, a reverse transcriptase, E. coli DNA polymerase, Klenow fragment polymerase, T7 RNA polymerase, T3 RNA polymerase, viral replicase, SP6 RNA polymerase, and combinations thereof. Preferably, the buffer is selected from the group consisting of T4 DNA ligase buffer and T4 RNA ligase buffer, and combinations thereof. Preferably, the ligase is selected from the group consisting of E. coli DNA ligase, T4 DNA ligase, and T4 RNA ligase, and combinations thereof. Preferably, the triphosphate mix is selected from the group consisting of dNTP and rNTP.

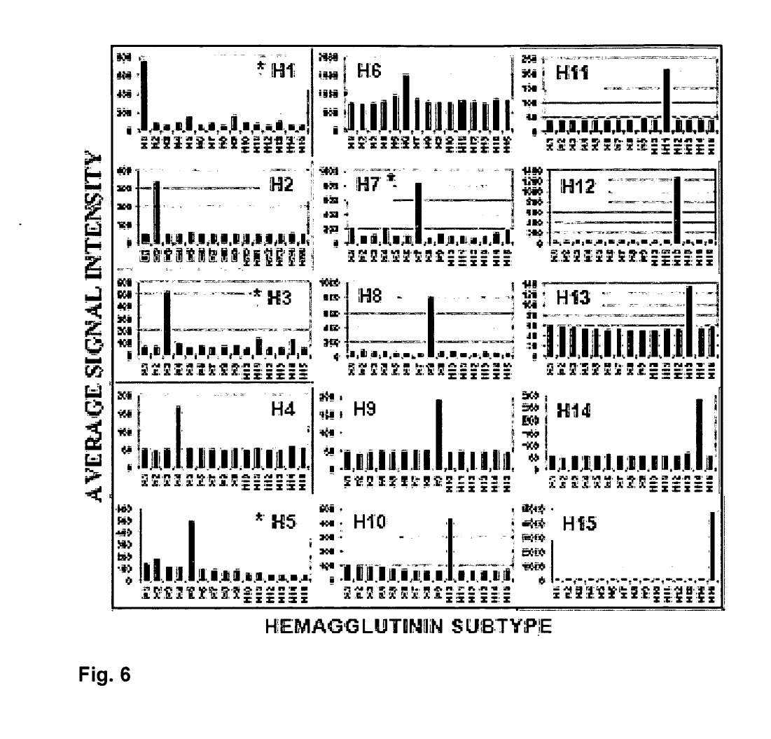

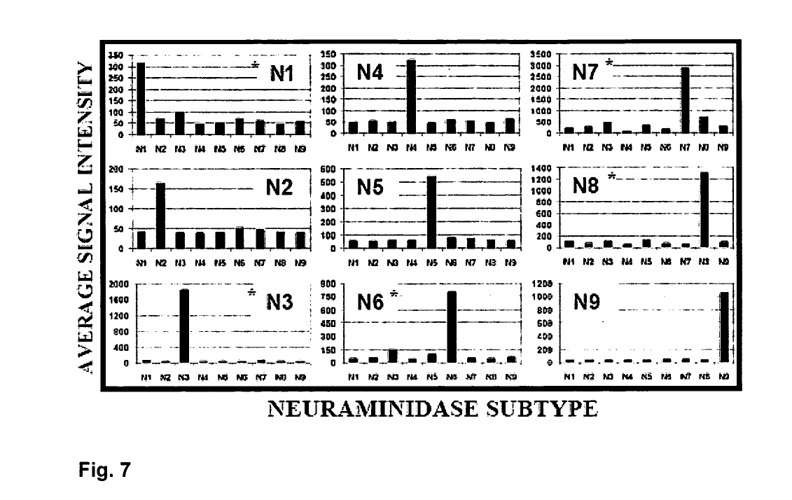

The results of subtyping samples of all subtypes of influenza A are shown in FIGS. 6 and 7. The starred subtypes are those that were done using straight hybridization. The subtypes without a star were done using the extension-ligation method.

What is claimed is:

1. A microarray device for genetic identification of upper respiratory pathogens comprising: a plurality of oligonucleotide probe sequences, wherein the oligonucleotide probe sequences correspond to distinct sequence regions of at least five pathogens selected from the group consisting of influenza A, influenza B, parainfluenza virus, adenovirus, enterovirus, rhinovirus, human metapneumovirus, respiratory syncytal virus, herpes simplex viruses, SARS coronavirus, Epstein-Barr virus, human herpes virus, pan bacteria, Chlamydia, Mycoplasma, streptococcus, Bacillus anthracis, Streptococcuspyogenes, Mycoplasmapneumoniae, Chlamydiapneumoniae, Bacillus thuringiensis, Bacillus subtilis, Bacillus cereus, and B. anthracis, and combinations thereof.

2. The microarray device of claim 1, wherein the oligonucleotide probe sequences for influenza A are selected from the group consisting of: SEQ ID NO:1-11, 251-261, 495-505, 726-736.

3. The microarray device of claim 1, wherein the oligonucleotide probe sequences for influenza B are selected from the group consisting of: SEQ ID NO:12-35, 262-285, 506-525, 737-758.

4. The microarray device of claim 1, wherein the oligonucleotide probe sequences for parainfluenza virus are selected from the group consisting of: SEQ ID NO:36-70, 286-315, 526-560, 759-792.

5. The microarray device of claim 1, wherein the oligonucleotide probe sequences for adenovirus are selected from the group consisting of: SEQ ID NO:71-95, 316-341, 561-582, 793-818.

6. The microarray device of claim 1, wherein the oligonucleotide probe sequences for enterovirus are selected from the group consisting of: SEQ ID NO:96-121, 342-368, 583-604, 819-842.

7. The microarray device of claim 1, wherein the oligonucleotide probe sequences for rhinovirus are selected from the group consisting of: SEQ ID NO:122-138, 369-385, 605-621, 843-859, and combinations thereof.

8. The microarray device of claim 1, wherein the oligonucleotide probe sequences for human metapneumovirus are selected from the group consisting of: SEQ ID NO:139-141, 386-388, 622-624, 860-862, and combinations thereof.

9. The microarray device of claim 1, wherein the oligonucleotide probe sequences for respiratory syncytal virus are selected from the group consisting of: SEQ ID NO:142-177, 389-425, 625-657, 863-896, and combinations thereof.

10. The microarray device of claim 1, wherein the oligonucleotide probe sequences for herpes simplex viruses are selected from the group consisting of: SEQ ID NO:178-184, 426-431, 658-665, 897-905, and combinations thereof.

11. The microarray device of claim 1, wherein the oligonucleotide probe sequences for SARS coronavirus are selected from the group consisting of: SEQ ID NO:185-202, 432-448, 666-682, 906-924, and combinations thereof.

12. The microarray device of claim 1, wherein the oligonucleotide probe sequences for Epstein-Barr virus are selected from the group consisting of: SEQ ID NO:203-204, 449, 925-927, and combinations thereof.

13. The microarray device of claim 1, wherein the oligonucleotide probe sequences for human herpes virus are selected from the group consisting of: SEQ ID NO:205-206, 450-451, 863, 928-929, and combinations thereof.

14. The microarray device of claim 1, wherein the oligonucleotide probe sequences for pan bacteria are selected from the group consisting of: SEQ ID NO:207-210, 452-454, 684-687, 930-933, and combinations thereof.

15. The microarray device of claim 1, wherein the oligonucleotide probe sequences for Chlamydia are selected from the group consisting of: SEQ ID NO:211-226, 455-470, 688-701, 934-949, and combinations thereof.

16. The microarray device of claim 1, wherein the oligonucleotide probe sequences for Mycoplasma are selected from the group consisting of: SEQ ID NO:227-238, 471-482, 702-713, 950-962, and combinations thereof.

17. The microarray device of claim 1, wherein the oligonucleotide probe sequences for streptococcus are selected from the group consisting of: SEQ ID NO:239-244, 483-488, 714-719, 963-968, and combinations thereof.

18. The microarray device of claim 1, wherein the oligonucleotide probe sequences for Bacillus anthracis are selected from the group consisting of: SEQ ID NO:245-250, 489-494, 720-725, 969-997, and combinations thereof.

19. The microarray device of claim 1, wherein the oligonucleotide probe sequences for Streptococcuspyogenes are selected from the group consisting of: SEQ ID NO:975-1013, and combinations thereof.

20. The microarray device of claim 1, wherein the oligonucleotide probe sequences for Mycoplasmapneumoniae are selected from the group consisting of: SEQ ID NO:1014-1051, and combinations thereof.

21. The microarray device of claim 1, wherein the oligonucleotide probe sequences for Chlamydiapneumoniae are selected from the group consisting of: SEQ ID NO:1052-1083, and combinations thereof.

22. The microarray device of claim 1, wherein the oligonucleotide probe sequences for Bacillus thuringiensis are selected from the group consisting of: SEQ ID NO:1084-1115, and combinations thereof.

23. The microarray device of claim 1, wherein the oligonucleotide probe sequences for Bacillus subtilis are selected from the group consisting of: SEQ ID NO:1116-1154, and combinations thereof.

24. The microarray device of claim 1, wherein the oligonucleotide probe sequences for Bacillus cereus are selected from the group consisting of: SEQ ID NO:1155-1157, and combinations thereof.

25. The microarray device of claim 1, wherein the oligonucleotide probe sequences for B. anthracis are selected from the group consisting of: SEQ ID NO:1158-1173, and combinations thereof.

26. The microarray device of claim 1, wherein the oligonucleotide probe sequences are selected from a 400 to 800 bases range corresponding to a unique gene selected from each pathogen.

27. The microarray device of claim 26, wherein the oligonucleotide probe sequences are selected from 400 to 800 bases range corresponding to the unique gene of each pathogen from a 5′ end.

28. The microarray device of claim 1, wherein the oligonucleotide probe sequences are attached to known locations by in situ electrochemical synthesis.

29. The microarray device of claim 28, wherein an electrode is associated with each of the known locations, wherein the electrodes are used to synthesize the oligonucleotide probe sequences.

30. The microarray device of claim 29, wherein the electrodes are on a solid surface having the known locations or on an opposing solid surface to the solid surface having the known locations.

31. The microarray device of claim 1, wherein the oligonucleotide probe sequences are attached to the known locations by a method selected from the group consisting of spotting, ink-jet printing, electric field deposition, and in situ photolithography synthesis.

32. A microarray device for subtyping influenza A comprising: a microarray device having a plurality of oligonucleotide probe sequences for subtypes of influenza A selected from the group consisting of at least five of Influenza A subtypes of H1, H2, H3, H4, H5, H6, H7, H8, H9, H10, H11, H12, H13, H14, H15, N1, N2, N3, N4, N5, N6, N7, N8, and N9, and combinations thereof.

33. The microarray device of claim 32, wherein the probe sequences that correspond the each subtype of influenza A as follows: H1 (SEQ ID NO:1174-1573), H2 (SEQ ID NO:1574-1973), H3 (SEQ ID NO:1975-2373), H4 (SEQ ID NO:2374-2573), H5 (SEQ ID NO:2574-2973), H6 (SEQ ID NO:2974-3369), H7 (SEQ ID NO:3370-3769), H8 (SEQ ID NO:3770-3887), H9 (SEQ ID NO:3888-4287), H10 (SEQ ID NO:4288-4390), H11 (SEQ ID NO:4391-4486), H12 (SEQ ID NO:4487-4587), H13 (SEQ ID NO:4588-4705), H14 (SEQ ID NO:4706-4761), H15 (SEQ ID NO:4762-4807), N1 (SEQ ID NO:4808-5207), N2 (SEQ ID NO:5208-5607), N3 (SEQ ID NO:5608-5878), N4 (SEQ ID NO:5879-5920), N5 (SEQ ID NO:5921-5995), N6 (SEQ ID NO:5996-6116), N7 (SEQ ID NO:6117-6216), N8 (SEQ ID NO:6217-6561), and N9 (SEQ ID NO:6562-6661).

34. The microarray device of claim 32, wherein the oligonucleotide probe sequences are selected from a 400 to 800 bases range corresponding to a unique gene selected from each pathogen.

35. The microarray device of claim 34, wherein the oligonucleotide probe sequences are selected from the 400 to 800 bases range corresponding to the unique gene from each pathogen at a 5′ end.

36. The microarray device of claim 32, wherein the oligonucleotide probe sequences are attached to known locations by in situ electrochemical synthesis.

37. The microarray device of claim 36, wherein an electrode is associated with each of the known locations, wherein the electrodes are used to synthesize the oligonucleotide probe sequences.

38. The microarray device of claim 37, wherein the electrodes are on a solid surface having the known locations or on an opposing solid surface to the solid surface having the known locations.

39. The microarray device of claim 32, wherein the oligonucleotide probe sequences are attached to the known locations by a method selected from the group consisting of spotting, ink-jet printing, electric field deposition, and in situ photolithography synthesis.

40. A pool of PCR primers for amplifying influenza A samples comprising: a PCR primer set selected from the group consisting of at least ten primer sequences from SEQ ID NO:6662-6699 and combinations thereof.

41. The pool of PCR primers of claim 40, wherein the PCR primer set is selected from a 50 to 200 bases range of HA and NA genes.

42. The pool of PCR primers of claim 40, wherein the PCR primer set is selected from a range of 450 to 700 bases from a 5′ end of HA and NA genes.

43. A pool of PCR primers for amplifying influenza A samples comprising: a PCR primer set selected from the group consisting of at least ten primer sequences from SEQ ID NO:6700-6731 and combinations thereof.

44. The pool of PCR primers of claim 43, wherein the PCR primer set is selected from a 50 to 200 bases range of HA and NA genes.

45. The pool of PCR primers of claim 43, wherein the PCR primer set is selected from range of 450 to 700 bases from a 5′ end of HA and NA genes.
